(19) United States
(12) Patent Application Publication
(10) Pub. No.: US 2024/0293223 A1
(43) Pub. Date: Sep. 5, 2024

(54) METHODS AND IMPLANTABLE PROSTHESIS FOR RECONSTRUCTION AND/OR AUGMENTATION OF AN ANATOMICAL SHAPE

(71) Applicant: Davol Inc., Warwick, RI (US)

(72) Inventors: Ian K. Parker, Bristol, RI (US); Robert D. Rehnke, St. Petersburg, FL (US); Alice M. Connelli, Warwick, RI (US); Evan Gray, Richmond, RI (US); Thomas Pine, Warren, RI (US); Kerri Marchessault, Hopkinton, MA (US)

(73) Assignee: Davol Inc., Warwick, RI (US)

(21) Appl. No.: 18/571,977

(22) PCT Filed: Jun. 27, 2022
(86) PCT No.: PCT/US2022/035059

§ 371 Date: Dec. 19, 2023

Related U.S. Application Data
(60) Provisional application No. 63/217,054, filed on Jun. 30, 2021.
(60) Provisional application No. 63/217,075, filed on Jun. 30, 2021.
(60) Provisional application No. 63/217,089, filed on Jun. 30, 2021.
(60) Provisional application No. 63/217,105, filed on Jun. 30, 2021.
(60) Provisional application No. 63/217,170, filed on Jun. 30, 2021.

(51) Int. Cl. A61F 2/12

(52) U.S. Cl. CPC A61F 2/12; A61F 2210/0004; A61F 2230/0093

(57) ABSTRACT

A prosthesis for reconstruction and/or augmentation of an anatomical shape. The prosthesis may have a three-dimensional configuration to reconstruct and/or augment the anatomical shape of a human breast. The prosthesis may be configured to encourage ingrowth of fat and/or tissue to fill open spaces within and about the prosthesis. The prosthesis may employ a structure having a plurality of cavities to allow fat and/or tissue to fill and pass through the structure and fill-out the anatomical shape of the reconstructed and/or augmented breast. The prosthesis may include a hollow core which may be accessible from one or both ends of the prosthesis. The prosthesis may be fabricated using a plurality of segments and/or layers of material which can be arranged about the core and joined to create the desired shape for the prosthesis.

### RELATED APPLICATIONS

Foreign priority benefits are claimed under 35 U.S.C. § 119(a)-(d) or 35 U.S.C. § 365(b) of U.S. application Nos. 63/217,054, filed Jun. 30, 2021, 63/217,075, filed Jun. 30, 2021, 63/217,089, filed Jun. 30, 2021, 63/217,105, filed Jun. 30, 2021, and 63/217,170, filed Jun. 30, 2021, each of which is incorporated herein by reference.

### FIELD

The present disclosure relates to an implantable prosthesis, and more particularly to a prosthesis for reconstruction and/or augmentation of an anatomical shape, including a human breast.

### BACKGROUND

Breast reconstruction is primarily performed following breast cancer diagnosis and surgical treatment. However, a growing number of patients are choosing breast reconstruction as a prophylactic option in response to genetic testing results which may indicate an individual being at high risk for breast cancer.

Breast reconstruction can be generally categorized as autologous and non-autologous. For autologous reconstruction, a patient's own tissue is harvested from another part of their body and then used to reconstruct the breast. For non-autologous reconstruction, an artificial implant, such as a saline, silicone or gel implant, is employed to reconstruct the breast mound.

Autologous reconstruction generally involves harvesting a tissue flap from the abdominal region of a patient. This procedure can maintain vascular supply to the patient's tissue, and generally provides an aesthetically pleasing outcome for the patient. However, such a procedure can be time consuming, with possible microsurgery to reconnect the vascular supply, and require a relatively longer recovery time. It can also create functional deficits and weakness in the area from which the tissue has been removed. This technique may not be available to some patients who lack belly volume or cannot afford a reduction of muscle mass.

Non-autologous reconstruction, which involves a wide majority of breast reconstructive procedures, may employ single stage or intermediate reconstruction procedures, or dual stage reconstruction procedures. A mastectomy and reconstruction of the breast can be performed at the same time (single stage) or staged over multiple procedures (dual stage). In each procedure, a breast implant is typically placed below the pectoral muscle, i.e, sub-pectoral, to mask the implant from being seen through the skin, and cover the relatively stiff implant with muscle.

In a single stage procedure, breast tissue is completely dissected and removed after a small incision is made under the breast. The pectoral muscle is subsequently detached at its lower end and the sub-muscular plane is developed to create a sub-pectoral pocket with sufficient size to accommodate the implant. An acellular dermal matrix (ADM) is typically employed to reattach the muscle and add reinforcement under the implant.

Single stage procedures generally do not provide much control over the cosmetic outcome because it cannot be adjusted over time. These procedures could also potentially result in tissue necrosis should the implant be too large for the size of the sub-muscular pocket.

In a dual stage procedure, the initial surgical stage is similar to the single stage procedure. However, rather than placing an implant into the sub-muscular pocket, an ADM is initially placed in the pocket and followed by placement of a tissue expander. The ADM is manipulated as needed to accommodate the tissue expander and then fixated into place. Following the initial surgical stage, the tissue expander is filled over multiple post-surgical office visits to slowly expand the space below the pectoral muscle to create a pocket. Once a sufficiently sized pocket is formed, typically six months after the initial procedure, a second surgical procedure is performed to remove the expander and insert the breast implant in the sub-muscular pocket created by the expander.

A more recent trend in breast reconstruction involves pre-pectoral placement of an implant on top of the pectoral muscle to avoid creation of a sub-muscular pocket. During such procedures, the implant is typically wrapped completely with ADM rather than using the ADM as a sling which only partially covers the implant.

It has been reported that the breast is shaped by a three-dimensional, fibrofatty fascial system. Two layers of this system surround the corpus mammae and fuse together around and anchor it to the chest wall in a structure identified as the circum-mammary ligament (CML). The CML, which defines the perimeter of the breast, is a 3D, roughly circular structure composed of superficial fascia collagen fibers that encase a ring of fat and attach it to the deep fascia of the chest, as a circular zone of adherence.

It is an object of the disclosure to provide methods and a prosthesis for augmenting and/or reconstructing a breast.

### SUMMARY

The present disclosure relates to methods and an implantable prosthesis for augmenting and/or reconstructing an anatomical shape.

In one embodiment, an implantable prosthesis comprises a body of biocompatible material having a proximal end and a distal end spaced from the proximal end. The body includes a hollow core structure extending along a core axis, a plurality of body segments attached to the hollow core structure, and a plurality of cavities formed by the plurality of body segments. The hollow core structure includes a proximal opening at a proximal end thereof and a distal opening at a distal end thereof. The body segments are arranged circumferentially about the hollow core structure with each of the plurality of body segments extending in an outward radial direction away from the core axis. The cavities are arranged in the outward radial direction facing away from the hollow core structure.

In one embodiment, an implantable prosthesis comprises a body having a three-dimensional configuration of biocompatible material. The body includes a hollow core located along a core axis, a plurality of body segments arranged circumferentially about the hollow core, and a plurality of cavities formed by the plurality of body segments. The hollow core has a distal end and a proximal end with the distal end being closed to block access to the hollow core from the distal end and the proximal end including an opening to permit access to the hollow core from the proximal end. Each of the plurality of body segments extends in an outward radial direction away from the core axis. The plurality of cavities face in the outward radial direction away from the hollow core.

In one embodiment, a method is provided for fabricating an implantable prosthesis. The method comprises acts of: (a) attaching a plurality of body segments to a sheet of implantable, biologically compatible material; (b) after act (a), rolling the sheet into a tubular configuration to form a hollow core structure about a core axis, the plurality of body segments extending from the hollow core structure in an outward radial direction and being arranged circumferentially about the core axis; and (c) after act (b), securing the sheet in the tubular configuration.

In one embodiment, an implantable prosthesis comprises a body having a three-dimensional configuration of biocompatible material. The body includes a hollow core located along a core axis, a plurality of body segments arranged circumferentially about the hollow core, and a plurality of cavities formed by the plurality of body segments. Each of the plurality of body segments extend in an outward radial direction away from the core axis. Each body segment includes at least four body layers fabricated from a sheet of material, the sheet being folded about a first fold and a second fold transverse to the first fold. The plurality of cavities face in the outward radial direction away from the hollow core.

In one embodiment, a method is provided for fabricating an implantable prosthesis. The method comprises acts of: (a) providing a plurality of sheets of biocompatible material; (b) folding one of the plurality of sheets along a first fold line and along a second fold line transverse to the first fold line to form a body segment including at least four body layers; (c) repeating act (b) for each of the plurality of sheets to form a plurality of body segments; and (d) arranging the plurality of body segments circumferentially about a core axis to form an implantable body having a three-dimensional configuration with the body layers forming a plurality of cavities facing in an outward radial direction away from the core axis.

In one embodiment, an implantable prosthesis comprises a body having a three-dimensional configuration of biocompatible material, the body having a proximal end and a distal end opposite the proximal end. The body includes a plurality of body segments located between the proximal and distal ends of the body, and a plurality of cavities formed by the plurality of body segments. The plurality of body segments are arranged circumferentially about a longitudinal axis extending in a direction from the proximal end to the distal end. Each of the plurality of body segments extends in an outward radial direction away from the longitudinal axis and has an outer periphery extending from the proximal end to the distal end of the body to define an outer profile of the body. The plurality of cavities face in the outward radial direction away from the longitudinal axis. The plurality of body segments includes first and second body layers connected to each other at first and second connections located between the proximal end of the body and the longitudinal axis and spaced apart from each other in an axial direction extending from the proximal end toward the distal end. The plurality of body segments further includes third and fourth body layers connected to the first and second body layers. The third body layer is connected to the first body layer at a third connection located between the first and second connections in the axial direction, and the fourth body layer is connected to the second body layer at a fourth connection located between the first and second connections in the axial direction.

In one embodiment, an implantable prosthesis comprises a tissue infiltratable body of biocompatible material having a proximal end and a distal end spaced from the proximal end, the body extending along a longitudinal axis from the proximal end to the distal end. The body includes a plurality of cavities circumferentially arranged around the longitudinal axis and extending in an outward radial direction facing away from the longitudinal axis. The plurality of cavities are arranged in a first tier, a second tier and a third tier stacked along the longitudinal axis between the proximal end and the distal end. The second tier is located between the first tier and the third tier. Each of the first tier, the second tier and the third tier including at least first and second cavities. The first cavity in the first tier is aligned with the first cavity in the third tier along a first radial plane extending in a direction from the proximal end to the distal end of the body. The second cavity in the first tier is aligned with the second cavity in the third tier along a second radial plane extending in a direction from the proximal end to the distal end of the body. The first radial plane is circumferentially offset from the second radial plane. The cavities in the first tier have a first shape and the cavities in the third tier have a third shape with the first shape being different from the third shape.

In one embodiment, an implantable prosthesis comprises a tissue infiltratable body of biocompatible material having a proximal end and a distal end spaced from the proximal end, the body extending along a longitudinal axis from the proximal end to the distal end. The body includes a plurality of cavities circumferentially arranged around the longitudinal axis and extending in an outward radial direction facing away from the longitudinal axis. Each of the plurality of cavities extends along a corresponding cavity axis in a direction transverse to the longitudinal axis. The plurality of cavities are arranged in a first tier, a second tier and a third tier stacked along the longitudinal axis between the proximal end and the distal end, with the second tier being located between the first tier and the third tier. Each of the first tier, the second tier and the third tier includes at least two cavities. The cavity axis for each cavity in the first tier has a first angle relative to the proximal end of the body, the cavity axis for each cavity in the second tier has a second angle relative to the proximal end of the body, and the cavity axis for each cavity in the third tier has a third angle relative to the proximal end of the body. The first, second and third angles are different from each other.

The foregoing is a non-limiting summary of the disclosure. Other aspects, embodiments and/or features will become apparent from the following description.

Various embodiments of the disclosure may provide certain advantages and may overcome certain drawbacks of prior prostheses. Embodiments of the disclosure may not share the same advantages, and those that do may not share them under all circumstances.

### BRIEF DESCRIPTION OF DRAWINGS

Aspects of the disclosure are described below, by way of example, with reference to the accompanying drawings, and wherein:

FIGS. 1A-1F are schematic illustrations of a breast reconstruction procedure;

FIG. 2 is a top, perspective view of an implantable prosthesis according to one aspect;

FIG. 3 is a side view of the implantable prosthesis of FIG. 2;

FIG. 4 is a top view of the implantable prosthesis of FIG. 2;

FIG. 5 is a bottom view of the implantable prosthesis of FIG. 2;

FIG. 6 is a bottom, perspective view of the implantable prosthesis of FIG. 2;

FIG. 7 is a top, perspective view of an implantable prosthesis according to one aspect;

FIGS. 8 and 9 are schematic illustrations of fabricating the body of the implantable prosthesis of FIGS. 2-7 according to one aspect;

FIGS. 10A-10C are schematic illustrations of fabricating a body segment of the implantable prosthesis of FIGS. 2-9 according to one aspect;

FIGS. 11-13 are schematic illustrations of a fabricated body segment of FIGS. 10A-10C;

FIG. 14 is a perspective view of the body of the implantable prosthesis schematically illustrating connections between the body segments according to one aspect;

FIG. 15 is a perspective view of the body of the implantable prosthesis schematically illustrating a distal layer connected to the distal end of the body to one aspect;

FIG. 16 is an enlarged view of FIG. 15 illustrating the connection between a tab at the distal layer and the body of the implantable prosthesis;

FIGS. 17 and 18 illustrate the body of the implantable prosthesis of FIGS. 2-7 with the cavities arranged in tiers;

FIG. 19 illustrates the body of the implantable prosthesis of FIGS. 2-7 with the cavities angled relative to the proximal end according to one aspect;

FIG. 20 illustrates the implantable prosthesis of FIGS. 2-7 covered with a shroud according to one aspect; and

FIG. 21 illustrates a preformed shroud according to one aspect.

### DESCRIPTION

It should be understood that aspects of the disclosure are described herein with reference to the figures, which show illustrative embodiments in accordance with aspects of the disclosure. The illustrative embodiments described herein are not necessarily intended to show all aspects of the disclosure, but are used to describe a few illustrative embodiments. Thus, aspects of the disclosure are not intended to be construed narrowly in view of the illustrative embodiments. It should be appreciated, then, that the various concepts and embodiments discussed herein may be implemented in any of numerous ways, as the disclosed concepts and embodiments are not limited to any particular manner of implementation. In addition, it should be understood that aspects of the disclosure may be used alone or in any suitable combination with other aspects of the disclosure.

The disclosure is directed to an implantable prosthesis for augmenting and/or reconstructing aa anatomical shape, although the prosthesis may be suitable for mending anatomical defects in, and weaknesses of, soft tissue and muscle walls or other anatomical regions. The phrase “mending a defect” includes acts of repairing, augmenting, and/or reconstructing a defect and/or a potential defect.

For ease of understanding, and without limiting the scope of the disclosure, the prosthesis is described below particularly in connection with breast reconstruction and/or augmentation. It should be understood, however, that the prosthesis is not so limited and may be employed in other anatomical procedures, as should be apparent to one of skill in the art. For example, and without limitation, the prosthesis, or aspects of the prosthesis, may be employed for hernias, chest or abdominal wall reconstruction, or large defects, such as those that may occur in obese patients. The prosthesis may include one or more features, each independently or in combination, contributing to such attributes.

As will be described further below, the prosthesis may have particular application with pre-pectoral breast reconstruction procedures.

### Pre-Pectoral Breast Reconstruction

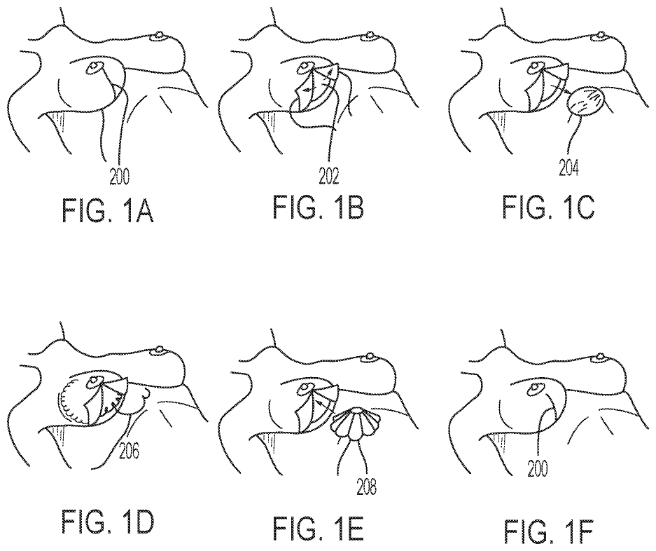

One embodiment of a pre-pectoral breast reconstruction procedure is illustrated in FIGS. 1A-1F

As illustrated in FIG. 1A, an initial incision 200 is made to form tissue flaps on the lower portion of the breast. The flaps 202 may be spread apart to provide access to the breast tissue and permit removal of a tumor or other growth, as illustrated in FIG. 1B. It is desirable to preserve skin and the nipple areola complex.

As illustrated in FIG. 1C, the corpus mammae 204 is removed, along with the bases of the Cooper's ligaments. Thereafter, as illustrated in FIG. 1D, the circum-mammary ligament (CML) may be tightened to reestablish a desirable diameter base for the breast. For example, the CML may be tightened to correspond with the breast's pre-op diameter base or tightened even more should the CML have been stretched and loosened over time due to aging and/or other factors.

As illustrated, the CML may be tightened using a purse string suturing technique in which a running suture 206 is placed around and/or through the CML and then pulled upwardly to cinch-up the tissue and tighten the base. However, other procedures are contemplated for reestablishing the base of the breast.

Once the CML has been tightened, a prosthetic implant 208 may be inserted, as illustrated in FIG. 1E, into the breast cavity created by the removal of the corpus mammae. Prior to insertion, the implant may be coated with a fat graft which has been lipo-aspirated from the patient. The fat graft may help soften the prosthesis and/or provide seeding for new fat and/or tissue being formed in and about the prosthesis. The fat graft may also reduce the potential for fluid to fill the space created by the removal of tissue during the procedure.

The fat graft may be harvested from the patient using standard lipo-aspiration techniques. The aspirated fat may be processed on site to remove oils and provide a more purified fat for the procedure. The processed fat may be applied to various surfaces and within various cavities of the prosthesis using a syringe or similar device, although other techniques are contemplated for coating the prosthesis.

Once inserted, the implant may be secured to the CML using sutures or other fasteners placed along the base of the device. Following implantation of the prosthesis, the incision 200 may be closed without tension, as illustrated in FIG. 1F.

Following the initial reconstruction procedure, additional fat grafting into the breast may be performed with one or more procedures over time to achieve a desired shape and/or feel for the breast and/or symmetry between both breasts.

### Prosthesis and Fabrication Concepts

The disclosure is more particularly directed to a prosthesis for reconstruction and/or augmentation of an anatomical shape. According to one aspect, the implantable prosthesis may have a three-dimensional configuration to reconstruct and/or augment the anatomical shape of a human breast. The prosthesis may be configured to encourage ingrowth of fat and/or tissue to fill open spaces within and about the prosthesis. The prosthesis may employ a body structure having a plurality of cavities to allow fat and/or tissue to fill and pass through the body structure and fill-out the anatomical shape of the reconstructed and/or augmented breast. The overall desired structure of the prosthesis may employ various constructs for its fabrication in an efficient manner.

According to one aspect, the prosthesis may be fabricated using a plurality of segments which can be arranged to create the desired shape for the prosthesis. In this manner, the individual segments may be fabricated in a relatively less complex manner and then assembled to create the desired cavities of the prosthesis. The segments may also be configured to provide the prosthesis with a desired amount of resilience and support.

According to one aspect, the implantable prosthesis may include multiple layers of material joined together to create the desired overall shape for the prosthesis. The layers may include one or more three-dimensional layers or structures which may be joined directly to each other or an adjacent two-dimensional layer of material. Each three-dimensional layer or structure may include multiple cavities, including three-dimensional cavities, for receiving fat and/or tissue.

According to one aspect, the prosthesis may employ one or more body segments configured to be expanded during fabrication of the prosthesis into a 3D structure having desired cavities. For example, and without limitation, a body segment may include a folded layer and/or multiple layers of material joined together so that expansion of each segment pulls the folds and/or layers apart to form 3D cavities for receiving fat and/or tissue. The expanded body segments may be joined in any suitable pattern to form cavities having desired shapes, sizes and/or arrangements. The body segments may be configured to form a body structure having a pleated, honeycomb-like, or other suitable 3D structure with tissue and/or fat-receiving cavities when expanded.

According to one aspect, each of the body segments may be connected to an adjacent body segment to form the cavities.

According to one aspect, a distal layer of material may be attached to the distal end of the body structure. The distal layer may be attached to one or more of the body segments. The body may include a hollow core. For some applications, the distal layer may overlic a distal opening of the hollow core to prevent access to the hollow core from the distal end thereof. For some applications, the distal layer may include an opening therethrough which is aligned with and permits access to the distal opening of the hollow core. The distal layer may have an annular configuration. For breast reconstruction and/or augmentation, the distal layer may be positioned to underlie and support a breast nipple and/or arcola.

According to one aspect, a proximal layer of material may be attached to the proximal end of the body opposite the distal end. The proximal layer may have an opening therethrough which is aligned with the proximal opening of the hollow core to permit access to the hollow core through the proximal layer. The proximal layer may have an annular configuration. The proximal layer may be attached to one or more of the body segments. For breast reconstruction and/or augmentation, the proximal layer may be positioned against the chest wall.

According to one aspect, each body segment may include a plurality of body layers extending in an outward radial direction away from the core axis. Each body segment may include multiple pairs of body layers coupled together. The body layers of each pair of body layers may be coupled together.

According to one aspect, the body may have a frusto-conical or semi-spherical shape.

According to one aspect, the body may be formed of absorbable material. For example, and without limitation, the body may be formed of P4HB (Poly-4-hydroxybutyrate).

According to one aspect, the body may be tissue infiltratable. The body may be formed of mesh fabric and the mesh fabric may be tissue infiltratable.

According to one aspect, the prosthesis may include multiple tiers or rows of cavities which are stacked relative to each other to create a desired arrangement of cavities.

Each tier may include cavities having the same shape, size, relative position and/or relative orientation. The cavities in one tier may be different from the cavities in another tier in terms of shape, size, position and/or orientation. Each tier may have a different size and/or shape relative to another tier.

Each tier may include cavities having the same angle relative to the proximal end of the body. The cavities in one tier may be angled differently as compared to another tier relative to the proximal end of the body.

For some applications, the prosthesis may have a configuration which may be trimmed, such as by a surgeon, to create a desired shape for implantation. For example, and without limitation, one or more layers and/or individual structures of the prosthesis may be trimmed to create a customized implant shape.

According to one aspect, the prosthesis may be provided with a proximal surface for placement against the pectoral muscle. The proximal surface may be planar or have a generally rounded shape including, but not limited to, a convex shape. Such a configuration may facilitate positioning and placement of the prosthesis against the pectoral muscle and within the circum-mammary ligament which establishes the position of the prosthesis on the chest wall. In this manner, the interaction between the implant and the anatomy may create a ball and socket-like arrangement.

According to one aspect, the prosthesis may include an outer shroud to cover the 3D body structure. The shroud may include a layer of fabric which is arranged over and attached to the body structure. If desired, the shroud may have a preformed 3D shape corresponding to the outer shape of the body. The shroud may cover the edges of the body segments to soften their interface with the anterior tissue flap. The shroud may promote rapid ingrowth to form a continuous layer of vascularized collagenated fibrous tissue that will support and promote growth of the adipose tissue layer above the prosthesis.

Various structural arrangements and/or manufacturing techniques may be employed to fabricate a relatively complex implant.

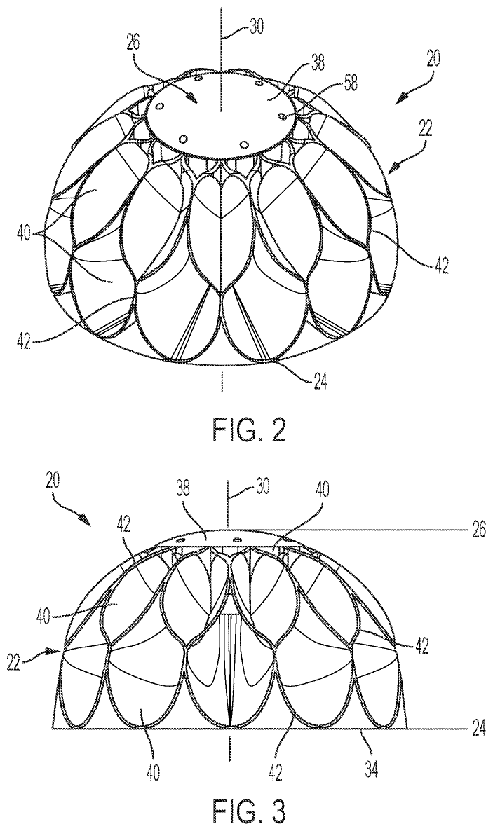

In one illustrative embodiment shown in FIGS. 2-6, the prosthesis 20 may include a body 22 configured to reconstruct and/or augment an anatomical shape. The body 22 may be configured to support the growth of tissue and/or fat as part of the reconstruction and/or augmentation.

In one embodiment, the body 22 may be formed with a three-dimensional configuration for reconstructing and/or augmenting the anatomical shape of a human breast. For example, and without limitation, the prosthesis may have a generally frusto-conical or hemispherical shape with a relatively larger proximal or lower end to be positioned adjacent the pectoral muscle of a patient and a relatively smaller distal or upper end opposite the lower end. The outer dimension of at least a portion of the body of the prosthesis may generally decrease in a direction from the proximal or lower end 24 toward the distal or upper end 26 of the prosthesis. If desired, the outer dimension of a portion of the body may initially remain constant before decreasing toward the distal end.

The outer profile of the body may be contoured with one or more segments to render a desirable configuration for the prosthesis. In one embodiment, the outer profile may be defined by multiple straight segments which are oriented at different angles relative to each other. In other embodiments, the outer profile of the body may be defined by a single curved segment, which is either convex or concave, multiple curved segments, a single straight segment, or a combination of curved segments and straight segments, as should be apparent to one of skill in the art to achieve a desired configuration.

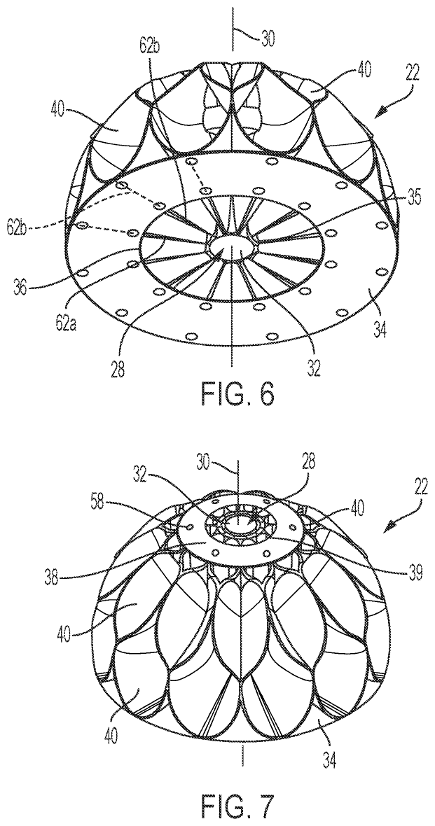

In one illustrative embodiment shown in FIGS. 5-6, the body may include a core 28 extending along a core axis 30 of the prosthesis. For some applications, it may be desirable to employ a hollow core. For example, and without limitation, a hollow core may allow fat and/or tissue to fill and pass through the body of the prosthesis. In another non-limiting example, a surgeon may pull a vascular flap pedicle along with blood vessels into one end of the hollow core.

According to one aspect, the core axis 30 may extend along the center of the body 22 with the hollow core centrally located within the body. In one embodiment, the body may employ a configuration which is symmetric about the core axis. However, it is to be appreciated that the body may employ an asymmetric configuration as should be apparent to one of skill.

According to one aspect, the core 28 may include a tubular structure having a wall 32 which defines the hollow core. Such an arrangement may provide a desired amount of column strength to the prosthesis for supporting the anatomical space being augmented or reconstructed. In one embodiment, the core 28 may be formed from a sheet of material which is rolled or otherwise shaped into a tubular configuration. However, the hollow core may be formed in any suitable manner as should be apparent to one of skill in the art. For example, and without limitation, the core may employ a length of prefabricated tubular material having a diameter suitable for a particular application.

For some applications, it may be desirable to provide one or more features to facilitate placement and/or attachment of the prosthesis at the surgical site. In one illustrative embodiment shown in FIGS. 2-6, the prosthesis may include a proximal layers or base 34 coupled to the proximal or lower end 24 of the body to facilitate placement and attachment of the prosthesis within the breast cavity. For example, and without limitation, the base may be configured to provide a support for attaching the prosthesis in position to the CML and/or adjacent tissue or muscle.

The proximal layer or base 34 may be configured to have any suitable shape and/or size conducive to positioning and/or fixating the prosthesis to adjacent tissue. For example, and without limitation, the base 34 may have a planar or non-planar configuration corresponding to the surgical site and/or suitable for a particular procedure employed for the reconstruction. In one embodiment, the base may be configured with a contoured shape to facilitate positioning and contact with adjacent tissue. For example, and without limitation, the base may have a curved contour, such as a convex curved outer surface, configured to fit within the CML, such as a ball-and-socket type arrangement. However, it is to be appreciated that the base may employ any suitable shape, either planar or non-planar, as should be apparent to one of skill.

The base 34 may be configured to be grasped and manipulated to position the prosthesis. One or more fasteners including, but not limited to, sutures, tacks and staples, may be employed attach the base to underlying muscle and/or tissue. The base may be formed from a layer of biocompatible material, including the material used for the body of the prosthesis.

For some applications, the base 34 may include a plurality of grips (not shown) protruding from a lower surface thereof which are configured to penetrate and grip adjacent tissue and/or muscle to facilitate positioning and/or fixation of the prosthesis.

For some applications, the base may include a support to help retain the base in an open, expanded configuration. For example, and without limitation, the support may include a resilient ring extending about the base at or in proximity to the outer periphery of the base. The support may be formed of a resorbable material so that it is resorbed by the body over time, although the support may be formed of non-resorbable material, if desired. In one embodiment, the support may be formed of a resorbable material and surrounded with a containment sleeve configured to contain the support as it breaks down while being resorbed.

For some applications, it may be desirable to gain access to the core 28 of the prosthesis. According to one aspect, a proximal end of the core at the proximal or lower end 24 of the body may include a proximal core opening 35 to allow access to the hollow core. In one embodiment, the proximal layer or base 34 may have an annular configuration with an inner opening 36 which is aligned with the proximal core opening 35 and/or the core axis 30 of the core. As shown in FIGS. 5-6, the inner opening 36 of the base may be larger than the core 28 so that proximal or lower portions of the body located radially outside the core may be exposed to adjacent tissue upon implantation of the prosthesis. However, it is to be appreciated that the base opening may have the same size or be smaller than the core, if desired.

The prosthesis may, additionally or alternatively, include a distal or upper layer 38 of material coupled to the distal or upper end 26 of the body opposite the proximal or lower end. The distal layer 38 may be configured to correspond with the region of a breast arcola and/or nipple when positioned within the breast cavity. The distal layer may be configured to support a breast areola and/or nipple, if present, when positioned within the breast cavity.

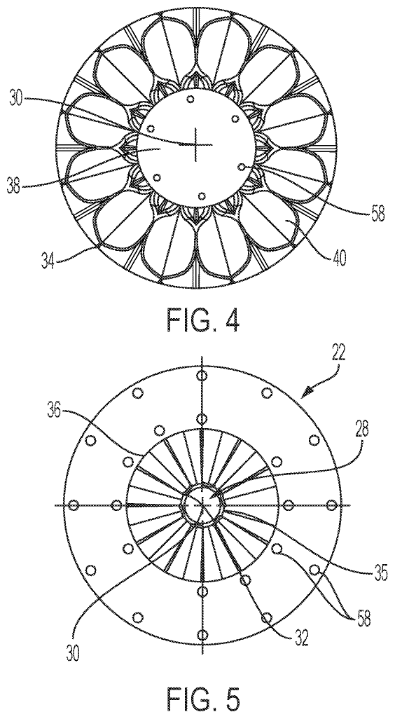

In one embodiment illustrated in FIG. 4, the distal layer 38 may be configured to completely overlie the distal end of the core at the distal or upper end 26 of the body to close off and prevent access to the hollow core 28 from the distal end 26 of the prosthesis. Such a configuration may be desirable for positioning below and to support a breast arcola and nipple. However, if desired, the distal layer of material may be provided with an opening therethrough which may be positioned to align with a distal core opening and provide access to the core from the distal or upper end of the prosthesis.

In one embodiment illustrated in FIG. 7, the distal layer 38 may have an annular configuration with an inner opening 39 which is aligned with the distal core opening and/or the core axis 30 of the core. As shown, the inner opening 39 of the distal layer may be larger than the core 28 so that distal or upper portions of the body located radially outside the core may be exposed to adjacent tissue upon implantation of the prosthesis. However, it is to be appreciated that the distal layer opening may have the same size or be smaller than the core, if desired.

As indicated above, the body 22 may be configured to support the growth of tissue and/or fat as part of the anatomical reconstruction. In one illustrative embodiment shown in FIGS. 2-7, the body 22 may include a plurality of outwardly facing cavities 40 which are arranged about the core axis 30 and surround the core 28. The cavities may be sized, shaped, arranged or otherwise configured to allow fat and/or tissue to fill and pass through the structure and fill-out the reconstructed and/or augmented breast. For some applications, it may be desirable, but not required, to place a selected amount of fat in the cavities prior to implantation of the prosthesis.

According to one aspect, the prosthesis body 22 may include a plurality of body segments 42 arranged to create a desired configuration of cavities. In one embodiment illustrated in FIGS. 2-7, the body segments 42 may be arranged circumferentially about the core 28 and extend in an outward radial direction from the core to form a petal-like configuration. The body segments 42 may be attached to each other and/or the core 28 to maintain positioning of the body segments and/or contribute to the structural integrity of the prosthesis. In this regard, the body segments 42 may be attached together and/or to the core 28 using any suitable technique as should be apparent to one of skill in the art. However, for some embodiments, it is to be understood that the body segments do not need to be attached to a hollow core structure. For example, the body may not include a hollow core structure and the body segments may be attached to each other about and spaced from the core axis to form the hollow core.

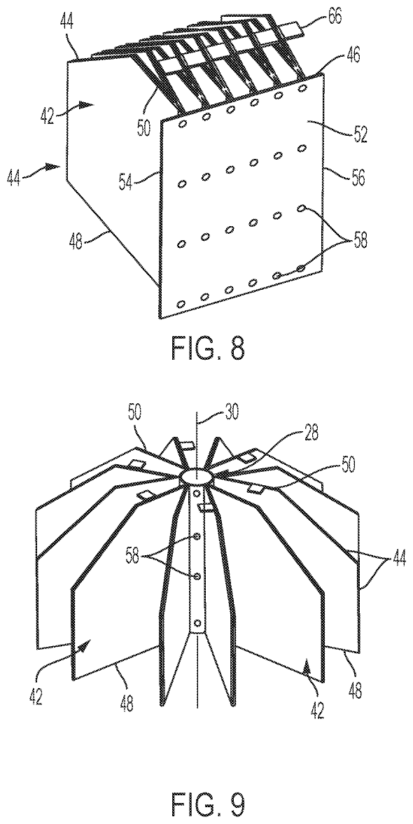

In one embodiment illustrated in FIGS. 8-9, each body segment 42 may have an outer periphery 44 extending from a proximal or lower end to a distal or upper end. The proximal and distal ends may correspond to the proximal and distal ends of the body. Each body segment may include an inner periphery 46 positioned along the core 28, a proximal or lower periphery 48 positioned at the proximal end 24 of the body, and a distal or upper periphery 50 positioned at the distal end 26 of the body. The outer periphery 44 may extend from the proximal periphery 48 to the distal periphery 50 of the body segment and have any suitable configuration to form a desired shape for the prosthesis.

In one embodiment, the inner periphery 46 of each body segment may be secured to the core with the outer periphery 44 being spaced in the radial direction away from the core.

In one illustrative embodiment shown in FIG. 8, the core 28 may be formed from a sheet or layer 52 of material. The body segments 42 may be attached to the core material at spaced apart locations relative to each other. As illustrated, each body segment 42 may be arranged with its inner periphery 46 extending across the length of the sheet of core material.

As illustrated in FIG. 9, after the body segments 42 have been attached to the sheet 52 of core material, the sheet 52 may be rolled into a tubular configuration about the core axis 30 which results in the body segments 42 being arranged about the core axis 30 in a fan-like arrangement with each body segment extending in an outward radial direction away from the core. Opposing ends 54, 56 of the sheet of core material may be overlapped and secured together to maintain the sheet in the tubular configuration and form the hollow core structure.

The body segments 42 may be attached to the sheet 52 of core material and the overlapping ends 54, 56 of the core material may be secured together at one or more locations. In one embodiment, four connections 58, such as an ultrasonic welds, may be employed to attach each body segment to the core material and the opposing ends of the core material together. The opposing ends of the core material may be overlapped and connected to each other. However, it is to be appreciated that any number of welds may be employed to achieve a desired level of attachment. Moreover, it is to be understood that the body segments and/or the core material may be secured using any suitable fastening technique as should be apparent to one of skill in the art.

According to one aspect of the disclosure, each body segment 42 may include multiple layers of material. The layers of material may be formed as a unitary structure or module which is connected to the core. Such an arrangement may facilitate fabrication of the prosthesis by reducing the number of separate components which need to be individually attached to the core. However, if desired, individual layers of material may be separately attached to the core as should be apparent to one of skill in the art.

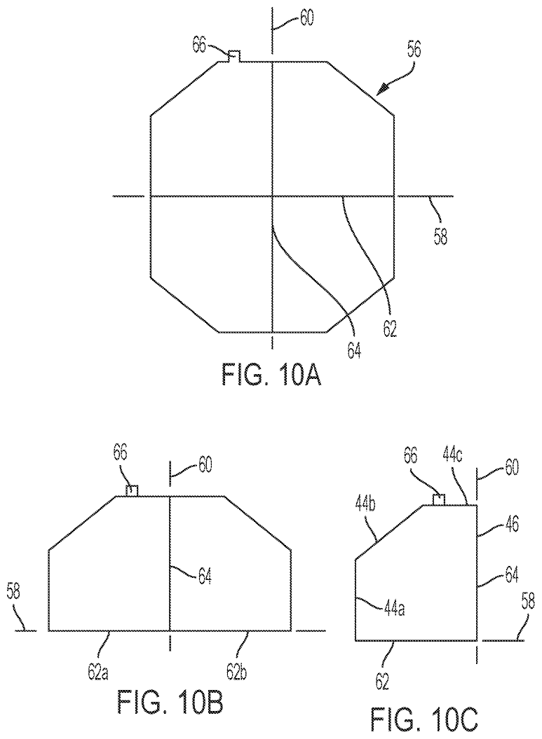

In one illustrative embodiment, each body segment 42 may include a plurality of body layers joined together as an integral structure. In one embodiment shown in FIGS. 10A-13, each body segment 42 may include four body layers 54 which are attached to the core 28 and extend in the outward radial direction. The body layers 54 for each body segment 42 may be arranged in two pairs 55a, 55b of body layers with each layer 54 of a pair of layers being joined to each other along their radially extending proximal peripheries 48. The body layers of each body segment may also be joined to each other along their inner peripheries 46 which extend in the axial direction along the length of the core.

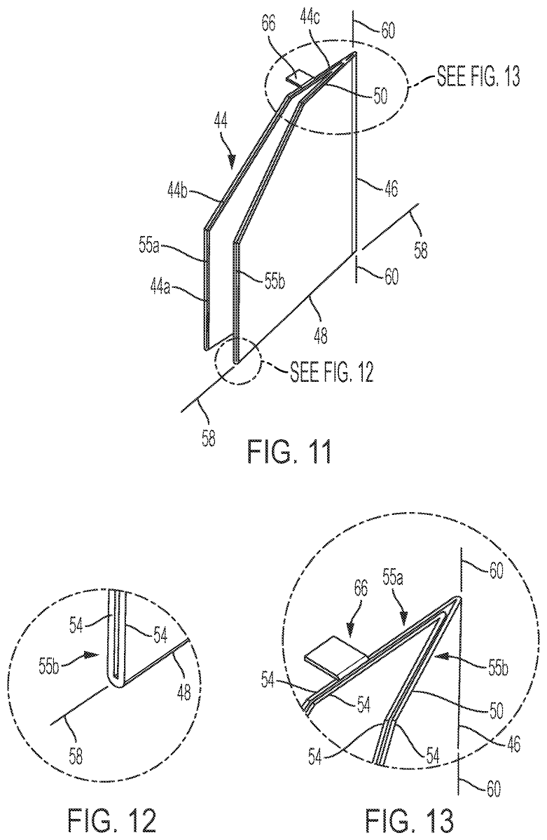

In one illustrative embodiment shown in FIGS. 10A-11, each body segment 42 may be formed from a single sheet 56 of material folded about a first axis 58 and a second axis 60 to form the four body layers. As shown in FIG. 10A, the sheet of material may be folded about the first axis 58 along a first fold 62 to form the proximal periphery 48 joining each pair of body layers. As shown in FIG. 10B, the sheet 56 of material may be subsequently folded about the second axis 60 along a second fold 64 to form the inner periphery 46 of the body layers. The second fold 64 divides the first fold 62 into first and second fold segments 62a, 62b. In one embodiment, the first and second folds 62, 64 may be perpendicular to each other. However, the sheet 56 of material may be folded along any number of folds oriented at any desired angles relative to each other to form the body segment with any suitable configuration and/or number of body layers as should be apparent to one of skill in the art.

As indicated above, the outer profile of the body 22 may be contoured to create a desirable configuration for the prosthesis. In one embodiment shown in FIG. 10A, the sheet of material forming the body segment may have an octagonal shape with each quadrant of the sheet forming one of the body layers of the body segment. As shown in FIG. 10C, the octagonal shape of the sheet results in each body layer having an outer periphery 44 with a first segment 44a, a second segment 44b and a third segment 44c which extend from the proximal end to the distal end of the body segment.

In one embodiment, the first segment 44a may be configured to extend a desired length in a direction parallel to the core axis 30 with the third segment 44c arranged to extend a desired length in a direction transverse to the first segment 44a and the core axis 30. The second segment 44b may be arranged to extend from the first segment 44a to the third segment 44c at an angle which is non-parallel and non-perpendicular to both the first and third segments. In this manner, the outer dimension of the first segment 44a may be constant relative to the inner periphery 46 in a direction from the proximal end toward the second segment 44b and the second segment may be angled so that its outer dimension decreases from the first segment 44a toward the third segment 44c located at the distal end 26 of the body.

In one embodiment, the third segment 44c may be arranged perpendicular to the first segment 44a, and the second segment 44b may be arranged at an angle of 35° to 40° relative to the proximal end 24 of the body segment. It is to be appreciated that the size and configuration of the sheet may be selected to form a body segment having body layers of any suitable size and shape for forming the body of the prosthesis as should be apparent to one of skill in the art. It is also to be understood that sheets of different sizes and/or configurations may be employed to form bodies having different sizes and configurations.

In one embodiment illustrated in FIGS. 10A-13, a tab 66 may extend from the third segment 44c of one or more of the body layers of each body segment. As discussed further below, the tab 66 may be employed for attaching a distal layer 38 to the body. However, it is to be appreciated that a tab is not required for each embodiment and/or the number of tabs may vary depending on a particular arrangement of an embodiment.

The cavities 40 of the body 22 may be formed by joining adjacent body layers 54 together in a manner which forms a desired configuration of cavities.

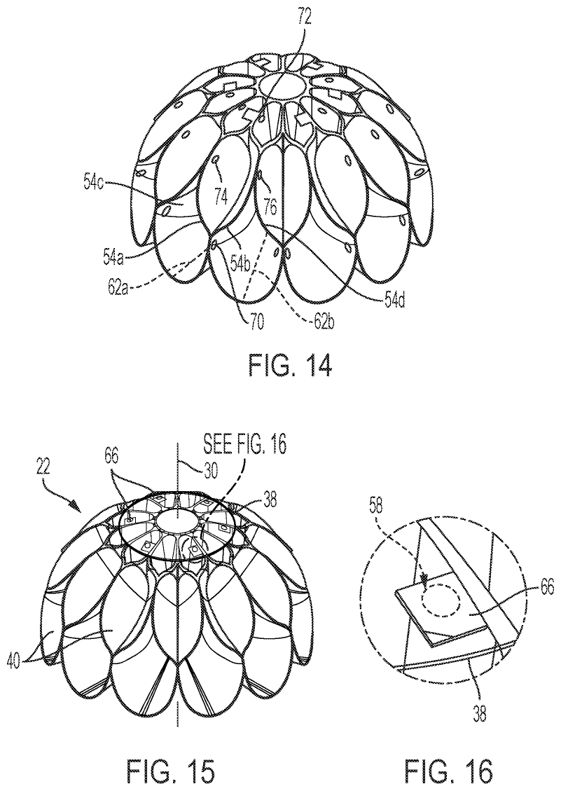

In one illustrative embodiment shown in FIG. 14, the body segments 42 may include first and second body layers 54a, 54b connected to each other at first and second connections 70, 72 located between the proximal end 24 and the distal end 26 of the body 22. The first and second connections 70, 72 may be spaced away from the core axis 30 and spaced apart in a proximal-to-distal direction extending from the proximal end toward the distal end of the body. In one embodiment, the second connection 72 may be located in close proximity to the distal end 26 of the body.

The body segments may also include third and fourth body layers 54c, 54d connected to the first and second body layers 54a. 54b. In one embodiment, the third body layer 54c may be connected to the first body layer 54a at a third connection 74 located between the first and second connections 70, 72 in the proximal-to-distal direction. The fourth body layer 54d may be connected to the second body layer 54b at a fourth connection 76 located between the first and second connections 70, 72 in the proximal-to-distal direction.

In one embodiment as shown in FIG. 14, each of the first, second, third and fourth connections may be located adjacent the outer periphery of the respective body layers. In one embodiment, the first, second, third and fourth connections are the only connections located between the proximal and distal ends of the body and between adjacent body layers. It is to be appreciated, however, that any number of connections may be employed between the body layers and located anywhere along the body layers to form a body having any suitable arrangement of cavities as should be apparent to one of skill in the art.

The first and third body layers 54a. 54c may be further connected together at the proximal end 24 of the body and the second and fourth body layers 54b, 54d may be further connected together at the proximal end 24 of the body. In one embodiment, the first and third body layers 54a. 54c may be connected together along the first fold segment 62a and the second and fourth body layers 54b, 54d may be connected together along the second fold segment 62b. As illustrated, each fold segment 62a, 62b may be oriented to extend in an outward radial direction away from the core axis and toward the outer periphery of the body layers.

The density and/or size of the cavities may be varied by the number and/or spacing of the connections between the layers. For example, placing the connections closer together may form relatively smaller and denser cavities, and spacing the connections further apart may form relatively larger and less dense cavities. If desired, a body structure may include regions having cavities with different sizes and/or densities by varying the patterns of the connections between the various layers of material.

In one embodiment, the body layers 54 may be connected together using welded connections, such as ultrasonic spot welds. However, it is to be appreciated that the body layers may be connected using any suitable fastening or joining technique including, but not limited to, sutures, staples and tacks, as should be apparent to one of skill in the art.

As described above, the implantable prosthesis may include a proximal layer 34 attached to the proximal end 24 of the body. In one embodiment illustrated in FIGS. 5 and 6, the proximal layer 34 may be attached to each of the first and second fold segments 62a, 62b. As illustrated, the proximal layer may be attached to the fold segments by a pair of connections 58, such as ultrasonic spot welds. However, it is to be appreciated that the proximal layer may be connected to the body using any suitable fastening or joining technique including, but not limited to, sutures, staples and tacks, as should be apparent to one of skill in the art.

The implantable prosthesis may include a distal layer 38 attached to the distal end 26 of the body. In one embodiment shown in FIGS. 15-16, the distal layer 38 may be attached to each tab 66 provided on the body segments. As illustrated, the distal layer may be attached to the tab by a connection 58, such as an ultrasonic spot weld. However, it is to be appreciated that the distal layer may be connected to the tabs using any suitable fastening or joining technique including, but not limited to, sutures, staples and tacks, as should be apparent to one of skill in the art.

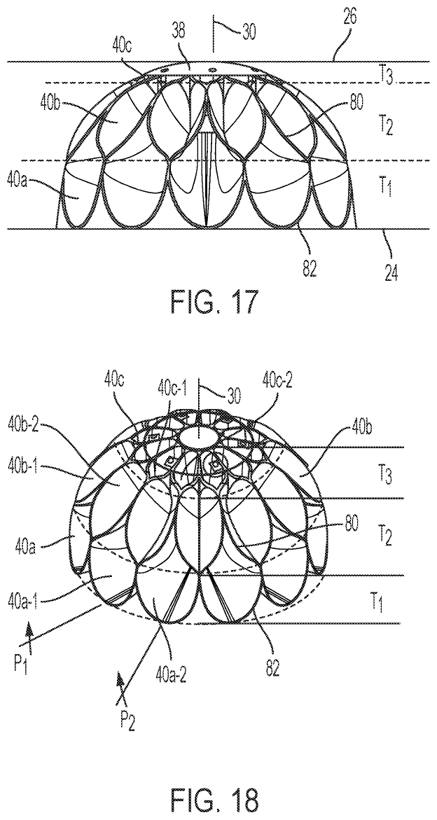

For some applications, it may be desirable to arrange the cavities in multiple tiers along the core axis. In one illustrative embodiment shown in FIGS. 17-18, the cavities 40 may be arranged in a first tier T₁, a second tier T₂ and a third tier T₃ stacked along the core axis 30 between the proximal end 24 and the distal end 26 of the body. The second tier T₂ may be located between the first tier T₁ and the third tier T₃. As shown in FIG. 18, eCA1ach tier may include at least first cavities 40a-1, 40b-1, 40c-1 and second cavities 40a-2, 40b-2 and 40c-2.

In one embodiment illustrated in FIG. 18, the first cavity 40a-1 in the first tier T₁ may be aligned with the first cavity 40c-1 in the third tier T₃ along a first radial plane P₁ extending in a direction from the proximal end to the distal end of the body. The second cavity 40a-2 in the first tier T₁ may be aligned with the second cavity 40c-2 in the third tier T₃ along a second radial plane P₂ extending in a direction from the proximal end to the distal end of the body. The first radial plane may be circumferentially offset from the second radial plane.

In one embodiment, the cavities 40b in the second tier T₂ may be circumferentially offset from the cavities 40a, 40c in the first and third tiers T₁, T₃. As illustrated in FIGS. 17-18, the cavities 40b in the second tier may separate the cavities 40a in the first tier from the cavities 40c in the third tier.

In one embodiment, each tier may include cavities having the same shape within the corresponding tier. In one embodiment, the cavities in the first tier T₁ may have a first shape and the cavities in the third tier T₃ may have a third shape which is different from the first shape. The cavities in the second tier T₂ may have a second shape which is different from the first and/or third shapes. However, it is to be appreciated that the cavities may have the same or different shapes within each tier and/or between the tiers.

In one embodiment illustrated in FIGS. 17-18, each cavity 40a in the first tier T₁ may include a periphery with a first portion 80 having a chevron shape and a second portion 82 having a curved shape. As illustrated, the second portion 82 of the periphery may have a concave curvature extending from the first portion 80 in a direction toward the distal end of the body. The first portion 80 of the periphery may be at least partially located between adjacent cavities in the second tier T₂.

In one illustrative embodiment shown in FIGS. 17-18, the cavities 40 may decrease in size in a direction of the core axis 30 from the proximal end 24 toward the distal end 26 of the body. Each of the first, second and third tiers of cavities may have an outer diameter which may decrease in the direction from the proximal end to the distal end. In one embodiment, the third tier T₃ may have an outer diameter which is smaller than the outer diameter of the first tier T₁. The second tier T₂ may have an outer diameter which is smaller than the outer diameter of the third tier T₃.

In one embodiment, the cavities provided in each tier may have the same size within the corresponding tier. Each cavity within a tier may have a size which increases in the outward radial direction away from the core axis toward the outer periphery of the body. In one embodiment, each cavity may have a width which increases in the outward radial direction. It is to be appreciated that the cavities in each tier may be configured to have different sizes as should be apparent to tone of skill in the art.

Each of the cavities 40 may extend along a corresponding cavity axis CA in a direction transverse to the core axis 30. The cavity axis CA extends along the length of the cavity 40 and is located approximately in the central region of the cavity. In one embodiment, the cavity axis for each of the cavities may be different relative to each other cavity. Each cavity axis may be oriented in a different direction relative to each other cavity. Each cavity axis may be angled relative to the proximal end 24 of the body.

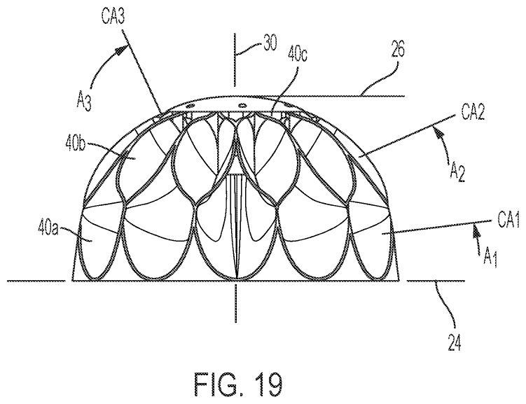

As shown in FIG. 19, the cavity axis CA1 for each cavity 40a in the first tier T₁ may have a first angle A₁, the cavity axis CA2 for each cavity 40b in the second tier T₂ may have a second angle A₂, and the cavity axis CA3 for each cavity 40c in the third tier T₃ may have a third angle A₃. In one embodiment, the first, second and third angles may be different from each other. For example, and without limitation, the first angle A₁ may be less than the second angle A₂ and/or the third angle A₃, and the second angle A₂ may be less than the third angle A₃. However, it is to be appreciated that the first, second and third angles may be the same angle, or any two of the first, second and third angles may be the same with the remaining angle being different from the other two angles.

In one embodiment, the first angle A₁ of the cavity axis CA1 may be the same for each cavity 40a in the first tier T₁. The second angle A₂ of the cavity axis CA2 may be the same for each cavity 40b in the second tier T₂. The third angle A₃ of the cavity axis CA3 is the same for each cavity 40c in the third tier T₃. However, it is to be appreciated that the cavity axes for the cavities in each tier do not need to have the same angle as each other and the angle of the cavity axes may vary within the corresponding tier.

The body 22 of the prosthesis may be configured to have any desired shape which may be suitable for a particular application. In one illustrative embodiment as shown in the figures, the body may have a frusto-conical shape which may be suitable for breast reconstruction and/or augmentation. Examples of other suitable configurations may include, but not be limited to a spherical shape, a semi-spherical shape and a tubular shape. As illustrated, the body may have a planar configuration at the proximal end 24 and/or the distal end 26. However, one or both of the proximal and distal ends may employ non-planar configurations including, but not limited to, curved configurations. For example, and without limitation, the proximal and/or distal ends may have a concave or convex shape.

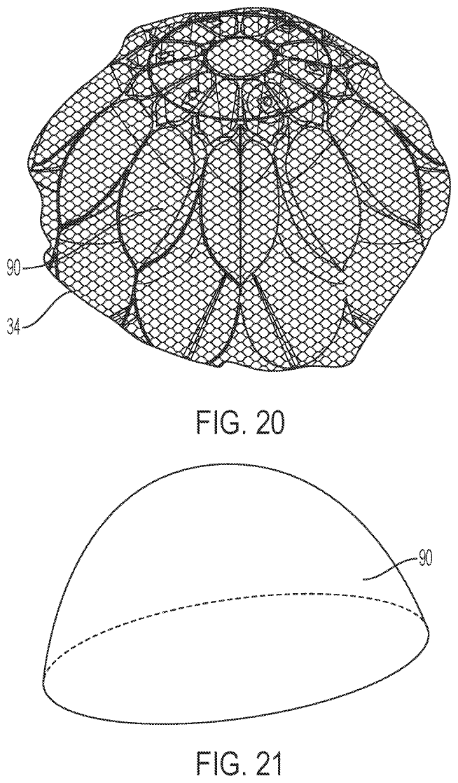

For some applications, it may be desirable to soften the interface between the body 22 of the prosthesis and adjacent tissue, such as the anterior tissue flap when used in pre-pectoral implant breast reconstruction or augmentation. In one illustrative embodiment shown in FIG. 20, a shroud 90 may be provided to cover the outer profile of the body. In one embodiment, the shroud 90 may include a flexible sheet of biocompatible material which is arranged over the body 22 and secured to at least the base 34 of the prosthesis. In one embodiment illustrated in FIG. 21, the shroud 90 may be preformed into a three-dimensional shape which corresponds to and fits over the body. For example, and without limitation, the shroud may be configured with a dome-like shape which may be secured to the base. In this manner, the shroud may cover the edges of the petals and/or body segments to soften their interface with adjacent tissue. The shroud may provide the prosthesis with a relatively smooth outer surface.

The shroud 90 may include a pliable mesh fabric which supports tissue ingrowth to create a continuous layer of vascularized collagenated fibrous tissue over the entire anterior profile of the prosthesis which will support and promote the growth of the adipose tissue layer above the prosthesis. In addition to softening the interface between the prosthesis, particularly edges of the petals and/or body segments, the shroud may reduce the need for autologous fat transfer (AFT) and/or may increase the additional volume created by AFT. The shroud may make the prosthesis less palpable sooner than a prosthesis without a shroud. The shroud may retain transferred adipose at the outer surface of the prosthesis and improve survivability of the adipose. The combined effect of softening edges and promoting adipogenesis may reduce the need for post-operative autologous fat grafting. However, it is to be appreciated that a shroud is not required for each embodiment of the prosthesis.

It may be desirable to provide a durable, light weight implantable prosthesis for breast reconstruction or augmentation without concern of long-term foreign material.

According to one aspect, the prosthesis may be fabricated from an absorbable material. In one embodiment, the prosthesis may be fabricated from a slow absorbing material, such as P4HB (Poly-4-hydroxybutyrate), to provide long term support for the breast as fat and/or tissue eventually fill and replace the prosthesis to promote a more natural appearance and feel for the breast. The material may be sufficiently porous to promote passage of fat and/or ingrowth of tissue within the prosthesis, although a porous material is not required for each embodiment. The prosthesis may include knitted, woven and/or non-woven material.

In one embodiment, the prosthesis, including the body and the shroud, if employed, may be fabricated with PHASIX mesh, which is manufactured from P4HB, available from Davol, Inc. of Warwick, RI. Other suitable materials may include, but are not limited to, GalaFLEX available from Galatea, TIGR Matrix available from Novus Scientific, SERI Surgical Body available from Allergen, BIO-A available from Gore, and ULTRAPRO available from Ethicon. If desired, a non-woven material, such as Phasix, may be employed as an alternative or together with a mesh to provide a relatively softer profile for the prosthesis. For some applications, it may be desirable to fabricate the prosthesis or one or more portions of the prosthesis from a non-absorbable material including, but not limited to, polypropylene and polytetrafluoroethylene (PTFE).

For embodiments employing a shroud which is formed over the body using a flat sheet of repair fabric, it may be desirbale to employ a relatively stretchable fabric as compared to a shroud which is preformed as a dome. For example, and without limitation, it may be desirable to employ a fabric having an amount of stretch which is approximately 50% greater than the stretch of a material used to preform the shroud as a dome. In one embodiment, GalaFlexLITE may be employed to form the shroud over and about the body. However, it is to be appreciated that other stretchable material may be employed to form the shroud as should be apparent to one of skill in the art.

For some applications, it may be desirable to coat the prosthesis with material to provide one or more properties. For example, and without limitation, it may be desirable to minimize bleeding, minimize seroma formation and/or facilitate tissue ingrowth. In one embodiment, the prosthesis may be coated with Arista AH available from Davol, Inc.

It may be desirable for particular applications for the prosthesis to be constructed to provide a desired amount of resistance to permanent deformation after compression. In one embodiment, the prosthesis may be constructed to have a reduction in height or no more than 10% (i.e., ≤10%) of its original height H after being subjected to vertical compression of 40% of its height H. The reduction in height may be determined at one or more times following compression. In one embodiment, the reduction in height may be determined at time t=0 and at time t=12 weeks. However, it is to be appreciated that the prosthesis may be configured to provide any suitable amount of resistance to permanent deformation as should be apparent to one of skill in the art.

It may be desirable to provide an implantable prosthesis which can support the resected space along with transferred adipose tissue during healing and integration of the implant.

According to one aspect, the prosthesis may have a compressive strength to oppose biomechanical forces within the breast. In one embodiment, the implant may have a compressive strength of at least 3.1 lbf (i.e., ≥3.1 lbf) at 25% vertical compression at time t=0 and at least 2.4 lbf (i.e., ≥2.4 lbf) at 25% vertical compression at time t=12 weeks. However, it is to be appreciated that the prosthesis may be configured to have any suitable amount of compressive strength as should appreciated by one of skill in the art.

According to one aspect, the prosthesis may employ connections having a connection strength which is sufficient to maintain the mechanical integrity of the device. In one embodiment, the implant may employ connections having a connection strength of at least 1.0 lbf (i.e., ≥1.0 lbf) at time t=0. However, it is to be appreciated that the prosthesis may be configured to have any suitable amount of connection strength as should appreciated by one of skill in the art.

For purposes of this patent application and any patent issuing thereon, the indefinite articles “a” and “an.” as used herein in the specification and in the claims, unless clearly indicated to the contrary, should be understood to mean “at least one.” The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

The use of “including,” “comprising,” “having,” “containing,” “involving,” and/or variations thereof herein, is meant to encompass the items listed thereafter and equivalents thereof as well as additional items.

It should also be understood that, unless clearly indicated to the contrary, in any methods claimed herein that include more than one step or act, the order of the steps or acts of the method is not necessarily limited to the order in which the steps or acts of the method are recited.

The foregoing description of various embodiments are intended merely to be illustrative thereof and that other embodiments, modifications, and equivalents are within the scope of the disclosure.

1. An implantable prosthesis comprising:
a body of biocompatible material having a proximal end and a distal end spaced from the proximal end, the body including:
a hollow core structure extending along a core axis, the hollow core structure including a proximal opening at a proximal end thereof and a distal opening at a distal end thereof;
a plurality of body segments attached to the hollow core structure, the plurality of body segments being arranged circumferentially about the hollow core structure, each of the plurality of body segments extending in an outward radial direction away from the core axis; and
a plurality of cavities formed by the plurality of body segments, the plurality of cavities arranged in the outward radial direction facing away from the hollow core structure.

2. The implantable prosthesis according to claim 1, wherein each of the plurality of body segments is connected to an adjacent body segment to form the plurality of cavities.

3. The implantable prosthesis according to claim 1, further comprising a distal layer of material attached to the distal end of the body.

4. The implantable prosthesis according to claim 3, wherein the distal layer is attached to one or more of the body segments.

5. The implantable prosthesis according to claim 4, wherein the distal layer overlies the distal opening of the hollow core to prevent access to the hollow core from the distal end thereof.

6. The implantable prosthesis according to claim 4, wherein the distal layer includes an opening therethrough which is aligned with the distal opening of the hollow core.

7. The implantable prosthesis according to claim 6, wherein the opening in the distal layer is larger than the distal opening of the hollow core.

8. The implantable prosthesis according to claim 6, wherein the distal layer has an annular configuration.

9. The implantable prosthesis according to claim 3, further comprising a proximal layer of material attached to the proximal end of the body opposite the distal end, the proximal layer having an opening therethrough which is aligned with the proximal opening of the hollow core to permit access to the hollow core through the proximal layer.

10. The implantable prosthesis according to claim 9, wherein the opening in the proximal layer is larger than the proximal opening of the hollow core.

11. The implantable prosthesis according to claim 9, wherein the proximal layer has an annular configuration.

12. The implantable prosthesis according to claim 9, wherein the proximal layer is attached to one or more of the body segments.

13. The implantable prosthesis according to claim 1, wherein each body segment includes a plurality of body layers extending in an outward radial direction away from the core axis.

14. The implantable prosthesis according to claim 13, wherein each body segment includes a plurality of pairs of body layers coupled together.

15. The implantable prosthesis according to claim 14, wherein the body layers of each pair of body layers are coupled together.

16. The implantable prosthesis according to claim 1, wherein the body has a frusto-conical shape.

17. The implantable prosthesis according to claim 1, wherein the body is formed of absorbable material.

18. The implantable prosthesis according to claim 17, wherein the body is formed of P4HB (Poly-4-hydroxybutyrate).

19. The implantable prosthesis according to claim 1, wherein the body is tissue infiltratable.

20.-22. (canceled)

23. The implantable prosthesis according to claim 1, wherein the body is configured to augment and/or reconstruct an anatomical shape of a human breast.

24.-40. (canceled)

41. An implantable prosthesis comprising:
a body having a three-dimensional configuration of biocompatible material, the body including:
a hollow core located along a core axis;
a plurality of body segments arranged circumferentially about the hollow core, each of the plurality of body segments extending in an outward radial direction away from the core axis, each body segment including at least four body layers fabricated from a sheet of material, the sheet being folded about a first fold and a second fold transverse to the first fold; and
a plurality of cavities formed by the plurality of body segments, the plurality of cavities facing in the outward radial direction away from the hollow core

42. The implantable prosthesis according to claim 41, wherein each of the body layers is coupled together along the second fold.

43. The implantable prosthesis according to claim 42, wherein the second fold extends in a direction parallel to the core axis.

44. The implantable prosthesis according to claim 41, wherein the second fold bifurcates the first fold into a first fold segment and a second fold segment, the first and second fold segments extending in a radial direction relative to the core axis.

45. The implantable prosthesis according to claim 44, wherein the first fold segment couples a first pair of the body layers together and the second fold segment couples a second pair of the body layers together.

46. The implantable prosthesis according to claim 45, wherein each of the first and second pairs of body layers includes an inner body layer and an outer body layer, the inner body layers of the first and second pairs of body layers facing each other and being located between the outer body layers of the first and second pairs of body layers.

47. The implantable prosthesis according to claim 46, wherein each body layer includes an outer periphery extending from a proximal end to a distal end of the body segment, the inner and outer body layers of each of the first and second pairs of body layers being attached to each other at a first location adjacent the outer periphery between the proximal end and the distal end of the body segment.

48. The implantable prosthesis according to claim 47, wherein the inner body layers of the body segment are attached to each other at a second location adjacent the outer periphery between the proximal end and the first location.

49. The implantable prosthesis according to claim 48, wherein each outer body layer of the body segment is attached the outer body layer of an adjacent body segment at a second location adjacent the outer periphery between the proximal end and the first location.

50. The implantable prosthesis according to claim 48, wherein each outer body layer of the body segment is attached to the outer body layer of an adjacent body segment at a third location between the first location and the distal end of the body segment.

51. The implantable prosthesis according to claim 47, wherein the first and second fold segments are located at the proximal end of the body.

52. The implantable prosthesis according to claim 51, further comprising a proximal layer of material attached to the proximal end of the body at the first and second fold segments of each body segment.

53. The implantable prosthesis according to claim 41, wherein the second fold is perpendicular to the first fold.

54. The implantable prosthesis according to claim 51, wherein the proximal end has a first diameter and the distal end has a second diameter which is smaller than the first diameter.

55. The implantable prosthesis according to claim 41, wherein the body has a frusto-conical shape.

56. The implantable prosthesis according to claim 41, wherein the body is formed of absorbable material.

57. The implantable prosthesis according to claim 56, wherein the body is formed of P4HB (Poly-4-hydroxybutyrate).

58. The implantable prosthesis according to claim 41, wherein the body is tissue infiltratable.

59.-61. (canceled)

62. The implantable prosthesis according to claim 41, wherein the body is configured to augment and/or reconstruct an anatomical shape of a human breast.

63. The implantable prosthesis according to claim 41, further comprising a hollow core structure located along the core axis to form the hollow core, the plurality of body segments being connected to the hollow core structure.

64.-181. (canceled)
